(19) United States
(12) Patent Application Publication
(10) Pub. No.: US 2019/0099173 A1
(43) Pub. Date: Apr. 4, 2019

(54) Implant Fixation with Suture Anchors

(71) Applicant: MED-EL Elektromedizinische Geraete GmbH, Innsbruck (AT)

(72) Inventors: Matthias Ladurner, Trins (AT); Andreas Müller, Gera (DE)

(21) Appl. No.: 15/722,005

(22) Filed: Oct. 2, 2017

(51) Int. Cl. A61B 17/04; A61B 17/06; A61B 17/34

(52) U.S. Cl. CPC A61B 17/0401; A61B 17/06166; A61B 17/24; A61B 2017/0446; A61B 2017/0445; A61B 17/3468

(57) ABSTRACT

A method is described for securing an implanted device within a patient. The overlying skin is lifted away from underlying bone at a selected device fixation location within the patient. Multiple suture anchor locations are then selected around or within the device fixation location. A suture anchor is installed at each of the suture anchor locations, the implanted device is placed at the device fixation location, and suture is connected to the suture anchors so as to securely fix the implanted device at the device fixation location.

### TECHNICAL FIELD

The present invention relates to medical implant systems such as implantable respiration pacing systems.

### BACKGROUND ART

It has become common that various medical devices and systems are implanted in a patient user. Such devices and their accessories and sensors need to be securely located within the body in a reliable manner. Otherwise these parts can migrate away from their intended location, creating problems such as lost connectivity to other components, sensors measuring the wrong values because of their improper positioning, and components being unable to operate as intended.

A variety of different ways of implant fixation are known, including no fixation at all, fixation within tissue using resorbable suture while hoping for tissue pocket formation in correct manner, fixation within tissue using non-resorbable suture, and periosteal fixation onto suitable bone locations.

### SUMMARY

Embodiments of the present invention are directed to a method for securing an implanted device within a patient. The overlying skin is lifted away from underlying bone at a selected device fixation location within the patient. Multiple suture anchor locations are then selected around or within the device fixation location. A suture anchor is installed at each of the suture anchor locations, the implanted device is placed at the device fixation location, and suture is connected to the suture anchors so as to securely fix the implanted device at the device fixation location.

In further specific embodiments, connecting suture to the suture anchors may include routing at least one length of suture to lie between the implanted device and the overlying skin and/or routing at least one length of suture between the implanted device and the underlying bone. Selecting the plurality of suture anchor location may include using a location template to determine the plurality of suture anchor locations. The underlying bone may be sternum bone, and the implanted device may be a laryngeal pacemaker.

### BRIEF DESCRIPTION OF THE DRAWINGS

FIG. 1A shows a top plan view of an implanted laryngeal pacemaker secured in place according to an embodiment of the present invention.

FIG. 1B shows a side cross-sectional view of the device of FIG. 1A.

FIGS. 2A-2F show stages in a process of securing an implanted device according to an embodiment of the present invention.

### DETAILED DESCRIPTION

Various embodiments of the present invention are directed to an improved method of implant fixation using commercially available suture anchors that conventionally have been used in orthopedics for performing standard procedures, typically to attach tendons to bone. Such suture anchors are provided by different manufacturers and are having different working mechanisms, but they all provide an anchoring mechanism that connects to human bone with attached sutures that are used to tighten the tendon to the bone. Suture anchors also are suitable to securely fix implantable medical devices to human bone. They can not only sustain high stresses over several weeks (as when used for connecting tendons to bone), but also for use for implant fixation over a period of many years.

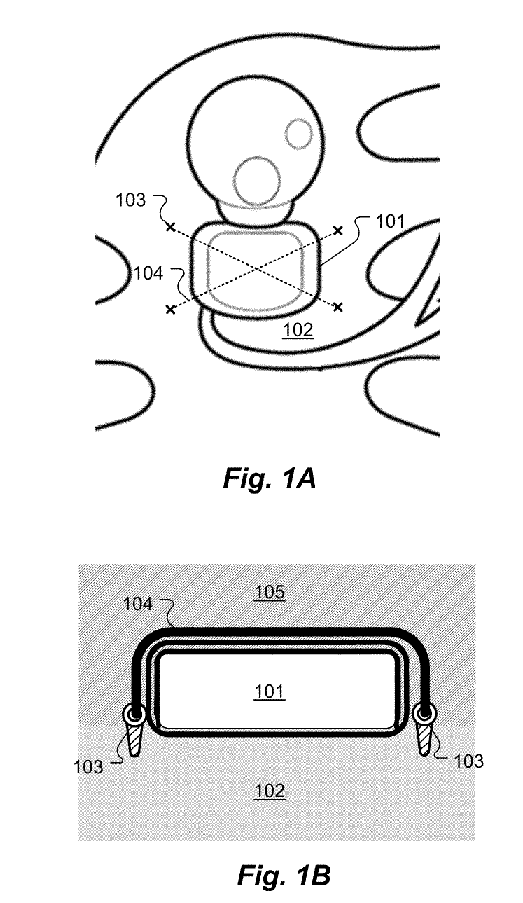

FIG. 1A shows a top plan view and FIG. 1B shows a corresponding side cross-sectional view of an implanted device, in this case a laryngeal pacemaker, which secured in place on the sternum bone according to an embodiment of the present invention. The overlying skin 105 is lifted away from underlying bone 102 at a selected device fixation location within the patient. The term Skin 105 in the context of the invention may include tissue, for example connective tissue, fascia and the periosteum or in general the tissue between the skin and bone 102. Multiple suture anchor locations are then selected around or within the device fixation location. A suture anchor 103 is installed at each of the suture anchor locations, the implanted device 101 is placed at the device fixation location, and non-resorbable suture 104 is connected to the suture anchors 103 so as to securely fix the implanted device 101 at the device fixation location. When the suture anchor locations are selected within the device fixation location, such selected suture anchor locations may lie under the overlapping implanted device 101 when fixated to bone 102 (not shown).

At least one length of suture 104 may be routed, at least partly, over the implanted device 101 as shown in FIGS. 1A and 1B to lie between the implanted device 101 and the overlying skin 105. In addition or alternatively, at least one length of suture 104 may be routed under the implanted device 101 between the implanted device 101 and the underlying bone 102.

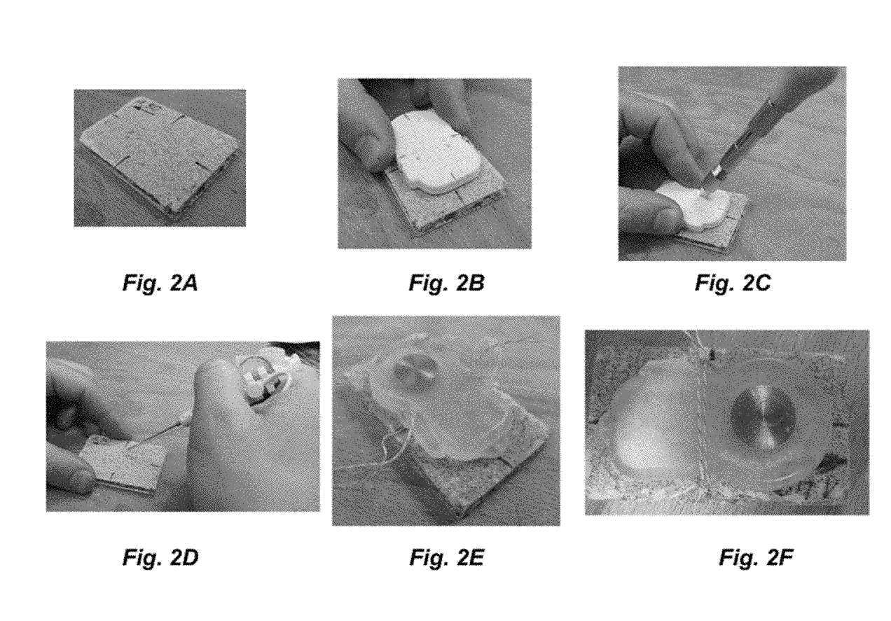

FIGS. 2A-2F show stages in a process of securing an implanted device according to one specific embodiment of the present invention. FIG. 2A shows a model of a sternum bone that was used as a target attachment location. FIG. 2B shows placement of a location template drill guide to determine the suture anchor locations at the attachment location. Suture anchor holes are created with a drill at the locations in the template as shown in FIG. 2C. The suture anchors are then inserted into the anchor holes as shown in FIG. 2D, and suture is routed through attachment holes in the suture anchors and on the housing of the implanted device, FIG. 2E. Finally, the suture is routed, tightened, and knotted to secure the implanted device to the bone, FIG. 2F.

Various exemplary embodiments of the invention have been disclosed, but it should be apparent that various changes and modifications can be made without departing from the true scope of the invention.

What is claimed is:

1. A method of securing an implanted device within a patient, the method comprising:
lifting overlying skin away from underlying bone at a selected device fixation location within the patient;
selecting a plurality of suture anchor locations around or within the device fixation location;
installing a suture anchor at each of the suture anchor locations;
placing the implanted device at the device fixation location; and
connecting suture to the suture anchors so as to securely fix the implanted device at the device fixation location.

2. The method according to claim 1, wherein connecting suture to the suture anchors includes routing at least one length of suture to lie between the implanted device and the overlying skin.

3. The method according to claim 1, wherein connecting suture to the suture anchors includes routing at least one length of suture between the implanted device and the underlying bone.

4. The method according to claim 1, selecting the plurality of suture anchor location includes using a location template to determine the plurality of suture anchor locations.

5. The method according to claim 1, wherein the suture is resorbable suture.

6. The method according to claim 1, wherein the suture is non-resorbable suture.

7. The method according to claim 1, wherein the underlying bone is sternum bone.

8. The method according to claim 1, wherein the implanted device is a laryngeal pacemaker.
